(19) United States
(12) Patent Application Publication
(10) Pub. No.: US 2010/0310600 A1
(43) Pub. Date: Dec. 9, 2010

(54) SELECTIVE AGONIST OF TOLL-LIKE RECEPTOR 3

(76) Inventors: William A. Carter, Spring City, PA (US); David Strayer, Bryn Mawr, PA (US)

Correspondence Address: NIXON & VANDERHYE, PC, 901 NORTH GLEBE ROAD, 11TH FLOOR, ARLINGTON, VA, 22203, US

(21) Appl. No.: 12/735,755

(22) PCT Filed: Feb. 17, 2009
(86) PCT No.: PCT/US2009/000959

§ 371 Date: Aug. 13, 2010

Related U.S. Application Data
(60) Provisional application No. 61/029,307, filed on Feb. 15, 2008.
(60) Provisional application No. 61/051,606, filed on May 8, 2008.

(51) Int. Cl. A61K 31/7105; A61K 39/12; A61K 39/02; A61K 39/00; A61K 39/002; A61P 35/04; A61P 33/00; A61P 31/04; A61P 31/12

(52) U.S. Cl. 424/204.1; 424/234.1; 424/265.1; 424/277.1; 424/93.7; 514/44 R

(57) ABSTRACT

A mismatched double-stranded ribonucleic acid, which is an agonist for Toll-like receptor 3 (TLR3), is used in vitro or in vivo as an antimicrobial agent, antiproliferative agent, and/or immunostimulant. Poly(l:C₁₁₋₁₄U) is a more selective agonist of TLR3 as compared to poly(l:C) even though the both double-stranded RNA are structurally analogous.

### CROSS-REFERENCE TO RELATED APPLICATION

This application claims the benefit of Application No. 61/029,307, filed Feb. 15, 2008, and Application No. 61/051,606, filed May 8, 2008.

### BACKGROUND OF THE INVENTION

This invention relates to providing an agonist for Toll-like receptor 3 (TLR3) for use as an anti-infectious agent (e.g., to treat or prevent an infection caused by at least one or more bacteria, protozoa, or viruses), an antiproliferative agent (e.g., to treat at least cancer, including virally-induced cancers), and/or an immunostimulant (e.g., to treat at least infectious disease or cancer by stimulating immunity, with or without vaccination). Methods of medical treatment and processes for manufacturing medicaments are provided.

Double-stranded RNA like poly(I:C) has been used as a TLR3 agonist. But its usefulness as a medicament is limited by its toxicity. Improved medicaments are thus sought that can be used as an anti-infectious agent, antiproliferative agent, and/or immunostimulant by specifically targeting TLR3, instead of other receptors belonging to this family. For example, a desirable medicament would have an increased therapeutic index (e.g., the ratio of the dose that produces a toxic effect divided by the dose that produces a therapeutic effect such as LD₅₀ divided by ED₅₀) for treating an incipient or established infection, treating a precancerous or cancerous condition, without inducing an excessive pro-inflammatory response as mediated by TLR3.

Double-stranded ribonucleic acid (dsRNA) triggers innate immunity (e.g., the production of host defenses) through dsRNA-dependent intracellular antiviral defense mechanisms including the 2′,5′-oligoadenylate synthetase/RNase L and p68 protein kinase pathways. But poly(I:C) also activates TLR3 and thereby induces secretion of proinflammatory chemokines and cytokines. See WO 2006/060513 at pages 3-4. This may initiate or enhance harmful inflammatory processes instead of selectively activating TLR3 to mediate the development of beneficial immunity. Poly(I:C) is believed to cause necrosis associated with inflammation, systemic inflammatory response syndrome, infection-associated acute cytokine storm, and chronic autoimmune diseases such as rheumatoid arthritis and inflammatory bowl disease. WO 2006/060513 taught that it would be beneficial to use a TLR3 antagonist as a medicament for various indications. Therefore, it was surprising that a selective agonist of TLR3 found use as a medicament in the present invention.

AMPLIGEN® poly(I:C₁₂U) from HEMISPHERx® Biopharma is a specifically-configured dsRNA with antiviral and immunostimulatory properties, but which exhibits reduced toxicity. AMPLIGEN® poly(I:C₁₂U) inhibits viral and cancer cell growth through pleiotropic activities: it regulates 2′,5′-oligoadenylate synthetase/RNase L and p68 protein kinase pathways as do other dsRNA molecules. We have now discovered that our specifically-configured dsRNA mediates its effects in the body by acting as a specific agonist of TLR3. Surprisingly, unlike other chemotherapeutic agents that are only effective against specific microbes (e.g., antibacterial penicillin, antiherpetic idoxuridine, and antimalarial chloroquine), we teach that poly(I:C₁₁₋₁₄U) has broad application as an antimicrobial chemotherapeutic agent effective in treatment of bacteria, viruses, and protozoa by acting directly on the immune system. Administration of poly(I:C₁₁₋₁₄U) avoids the side effects observed with poly(I:C) such as initiation or enhancement of harmful inflammatory processes.

It is an objective of the invention to provide treatment for a patient in need of an anti-infectious agent, antiproliferative agent, and/or immunostimulant. Our specifically-configured dsRNA is a more selective agonist of TLR3 as compared to poly(I:C) even though the two double-stranded RNA are structurally analogous. A long-felt need for a selective TLR3 agonist is addressed thereby. Methods for treating subjects and processes for making medicaments, especially involving infectious disease, cell proliferation, and/or vaccination, are provided. Further objectives and advantages are described below.

### SUMMARY OF THE INVENTION

The invention may be used to treat a subject (e.g., human or animal) with an incipient or established microbial infection, a pathological condition marked by abnormal cell proliferation (e.g., neoplasm or tumor), or as an immunostimulant to treat the subject for a disease or condition caused by at least infection, abnormal cell proliferation, or cell damage from autoimmunity or neurodegeneration. It is preferred that the amount of mismatched double-stranded ribonucleic acid (dsRNA) used is sufficient to bind Toll-Like Receptor 3 (TLR3) on immune cells of the subject. Innate or adaptive immunity may be triggered thereby. In particular, a specifically-configured dsRNA may be used to activate TLR3 without activating other Toll-like receptors like TLR4 or an RNA helicase like RIG-I or mda-5, or without inducing an excessive pro-inflammatory response as seen with poly(I:C), which is a nonselective TLR3 agonist.

A subject may be infected with at least one or more bacteria, protozoa, or viruses. A pharmaceutical composition which is comprised of specifically-configured dsRNA in an amount sufficient to bind to TLR3 is administered to the subject. Infection of the subject is reduced or eliminated thereby as assayed by decreased recovery time, increased immunity (e.g., increase in antibody titer, lymphocyte proliferation, killing of infected cells, or natural killer (NK) cell activity), decreased division or growth of the microbe, or any combination thereof as compared to the subject not treated with specifically-configured dsRNA. The immunity induced by treatment is preferably specific for the microbe.

A subject may be afflicted by abnormal cell proliferation (e.g., neoplasm or tumor, other transformed cells). A pharmaceutical composition which is comprised of specifically-configured dsRNA in an amount sufficient to bind to TLR3 is administered to the subject. Disease in the subject is reduced or eliminated thereby as assayed by improved morbidity or mortality, increased immunity (e.g., increase in antibody titer, lymphocyte proliferation, killing proliferating or transformed cells, or NK cell activity), decreased division or growth of proliferating or transformed cells, or any combination thereof as compared to the condition of a subject not treated with specifically-configured dsRNA.

Dendritic cell maturation may be induced in the subject. Immature dendritic cells, which are capable of antigen uptake, may be induced to differentiate into more mature dendritic cells, which are capable of antigen presentation and priming an adaptive immune response (e.g., antigen-specific T cells). During their conversion from immature to mature dendritic cells, they may at least change their cell-surface expression of major histocompatibility complex (MHC) molecules, costimulatory molecules, adhesion molecules, or chemokine receptors; decrease antigen uptake; increase secretion of chemokines, cytokines, or proteases; grow dendritic processes; reorganize their cytoskeleton; or any combination thereof. They may be induced to migrate to sites of inflammation or lymphoid tissue through blood or lymph to bring microbes, neoplastic or tumor cells, or other transformed cells into proximity.

A subject may be vaccinated against at least infection or cancer. Sometimes, e.g., virus-induced cancers, both infection and cancer may be treated. Immediately before, during, or immediately after vaccination (e.g., within 10 days of vaccination), a pharmaceutical composition which is comprised of specifically-configured dsRNA in an amount sufficient to bind to TLR3 is administered to the subject. The immune response to a vaccine or dendritic cell preparation is stimulated thereby. The vaccine or dendritic cell preparation may be comprised of killed, fixed, or attenuated whole microbes or cells (e.g., proliferating or transformed); a lysate or purified fraction of microbes or cells (e.g., proliferating or transformed); one or more isolated microbial antigens (e.g., native, chemically synthesized, or recombinantly produced); or one or more isolated tumor antigens (e.g., native, chemically synthesized, or recombinantly produced). In situ vaccination may be accomplished by the subject's production of antigen at a site or circulation thereto (e.g., produced in a natural infection or cell growth, or shed antigen), and specifically-configured dsRNA acting as an adjuvant thereon.

Antigen presenting cells (e.g., B lymphocyte, dendritic cell, macrophage) and mucosal tissues (e.g., gastric or respiratory epithelium) are preferred targets in the body for the specifically-configured dsRNA. The microbial or tumor antigen(s) may be presented, and the antigen(s) should be susceptible to the sole action of the specifically-configured dsRNA acting exclusively as a TLR3 agonist. Microbes, cancer cells, or other transformed cells may be susceptible to specific cytokine response patterns activated by specifically-configured dsRNA acting exclusively as a TLR3 agonist. The specifically-configured dsRNA is preferably administered by intravenous infusion; intradermal, subcutaneous, or intramuscular injection; intranasal or intratracheal inhalation; or oropharyngeal or sublingual application.

Also provided are processes for using and making medicaments. It should be noted, however, that a claim directed to the product is not necessarily limited to these processes unless the particular steps of the process are recited in the product claim.

Further aspects of the invention will be apparent to a person skilled in the art from the following description of specific embodiments and the claims, and generalizations thereto.

### BRIEF DESCRIPTION OF THE DRAWINGS

FIG. 1 shows that prime-boost immunization with 5 μg α-DEC-gag and 50 μg poly(I:C) provides protective immunity to airway challenge with vaccinia gag virus. (A) Average weight loss and (B) vaccinia plaque-forming titers in lung as mean±SD post challenge from six experiments: three each for BALB/c and C57BL/6 mice (except control Ig-p24; n=2 in BALB/c). Mice were primed and boosted at six week intervals with the indicated vaccine. Another group was immunized with α-DEC-p24 and poly(I:C) at the time of the boost only. Six to eight weeks after boost, mice were challenged intranasally with 5×10⁴ PFU vaccinia-gag. (C) Average weight loss and (D) vaccinia plaque-forming titers in lung as mean±SD post challenge from one of two similar experiments: five each for C57BL/6, DEC-205−/−, and TLR3−/− mice were immunized with α-DEC-p41. Six to eight weeks after boost, mice were challenged intranasally with 5×10⁴ PFU vaccinia-gag.

FIG. 2 shows that poly(I:C₁₂U) acts as an adjuvant for CD4+ T cell immunity to 5 μg α-DEC-p24 vaccine in a TLR3-dependent manner. (A) C×B6 F1 mice were injected intraperitoneally with α-DEC-p24 plus either graded doses of poly(I:C) or poly(I:C₁₂U) or PBS, then boosted with the same conditions six weeks later. The percentage of IFN-γ-producing and proliferating CD3+CD4+ T cells in response to HIV gag p17 or p24 mix one week after boost are shown as mean±SD (n=4 mice). (B) IFN-γ-secretion in response to HIV gag p24 peptides by CD4+ splenocytes in wild-type, TLR3−/−, and MDA5−/− mice immunized with two doses of α-DEC-p24 plus either 50 μg poly(I:C) or 250 μg poly(I:C₁₂U).

FIG. 3 shows that the HIV gag specific CD4+ T cell response after prime-boost immunization with α-DEC p24 and poly(I:C₁₂U) was characterized. C57BL/6 mice were injected subcutaneously with 5 μg of α-DEC-p24 and either 2, 10, or 50 μg poly(I:C₁₂U) or phosphate buffered saline (PBS), then boosted with the same conditions six weeks later. An additional group was immunized with 5 μg of α-DEC-p24 and 50 μg poly IC at the time of the boost only. Two weeks later, the frequency of IFN-γ+, TNF-α+, IL-2+, CD4+ T cells in gated CD3+ splenic T cells was analyzed in response to HIV gag p24 peptide mix. (A) Total frequency of IFN-γ−, TNF-α−, or IL-2-producing CD4+ T cells for each vaccine group. (B) The percentage of CD4+ T cells from the total cytokine response expressing all three cytokines (red), any two cytokines (blue), or any one cytokine (green), for each vaccine group is represented pictorially by pie charts. The total frequency of cytokine-producing CD4+ T cells is shown.

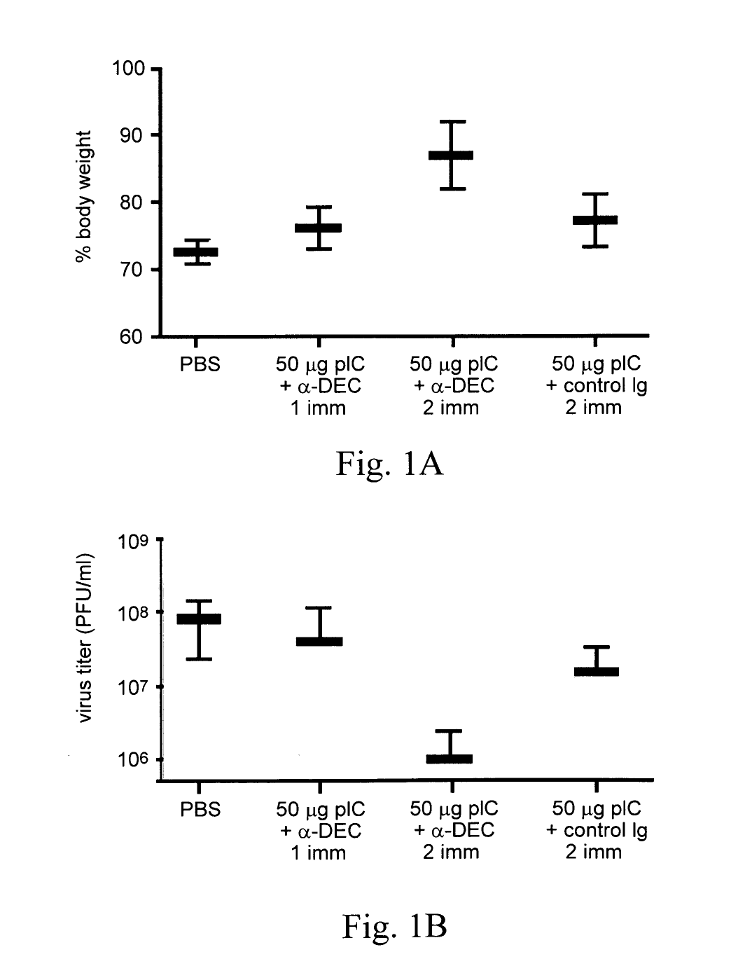

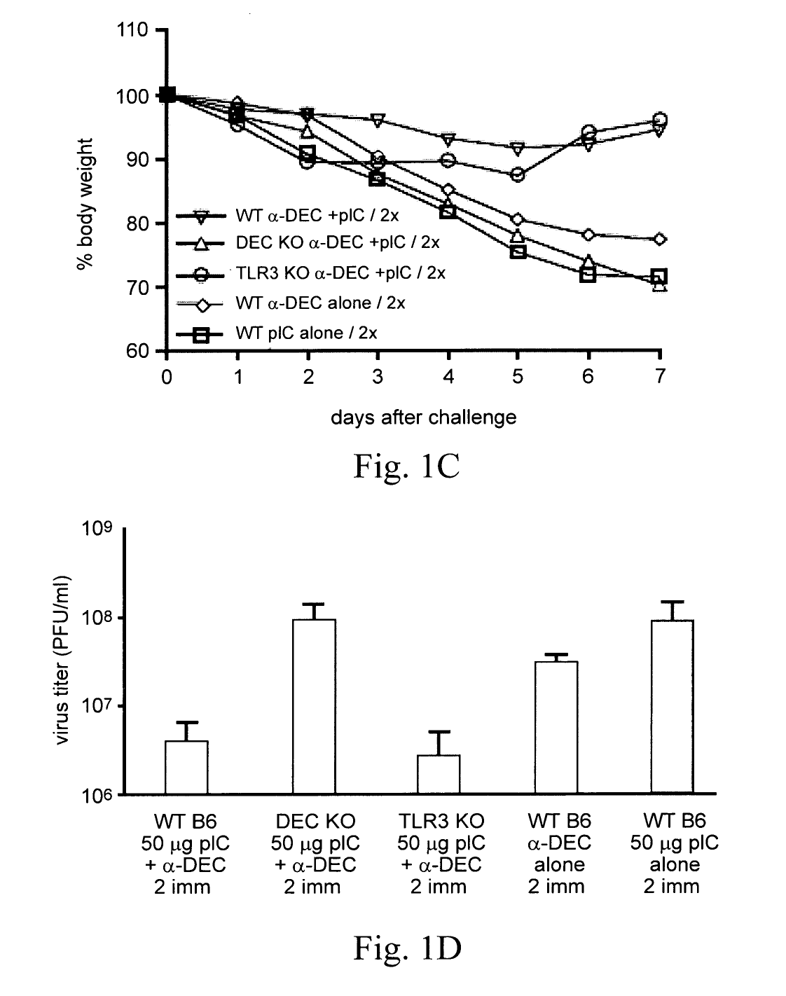

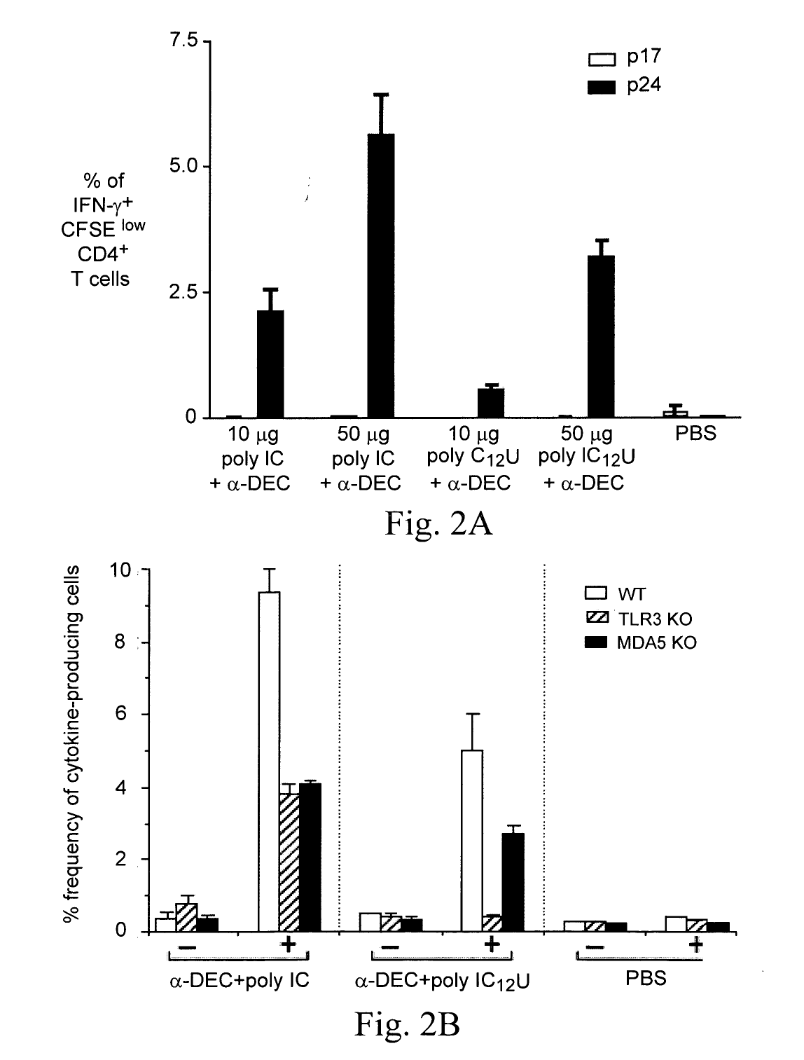

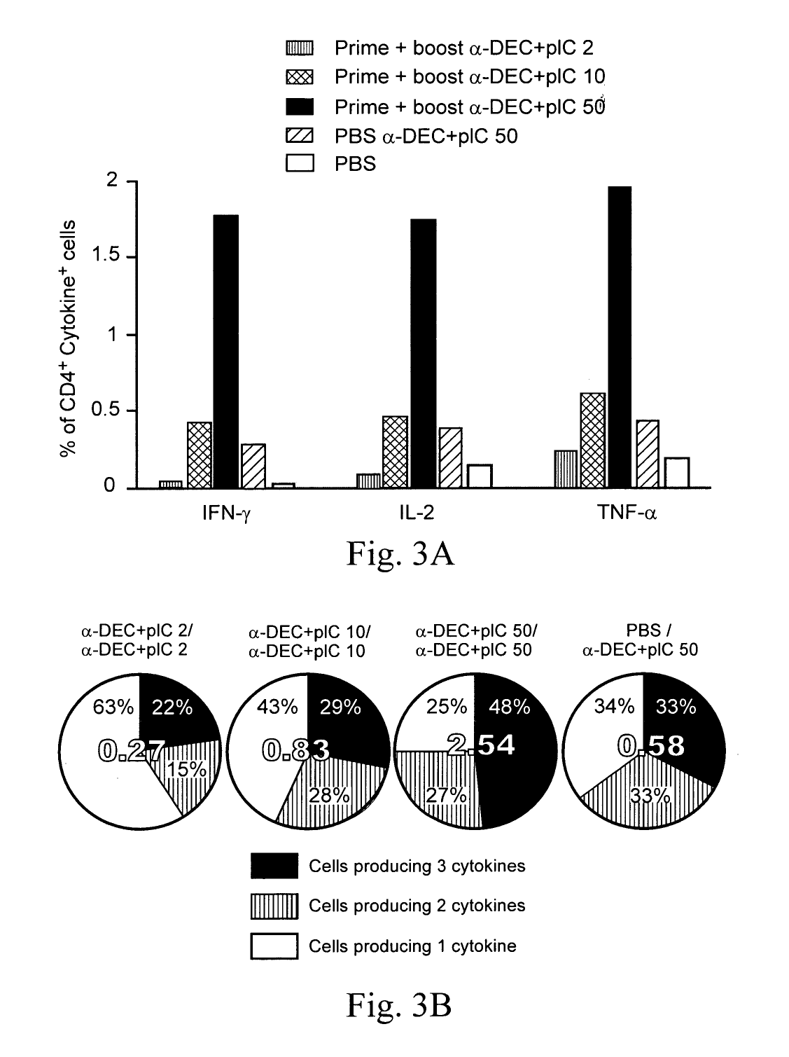

### DESCRIPTION OF SPECIFIC EMBODIMENTS OF THE INVENTION

An infection by a microbe may be treated. They may infect a human or animal subject. The infection may be incipient or established. The microbe may be a bacterium, protozoan, or virus; especially those that cause disease (i.e., pathogenic microbes). Here, the terms “microbe” and “microorganism” are used interchangeably.

The bacterium may be a species of the genus Bacillus (e.g., B. anthracis, B. cereus), Bartonella (B. henselae), Bordetella (e.g., B. pertussis), Borrelia (e.g., B. burgdorferi), Brucella (e.g., B. abortus), Campylobacter (e.g., C. jejuni), Chlamydia (e.g., C. pneumoniae), Clostridium (e.g., C. botulinum, C. difficile, C. perfringens, C. tetani), Corynbacterium (e.g., C. amycolatum, C. diphtheriae), Escherichia (e.g., E. coli O175:H7), Haemophilus (e.g., H. influenzae), Heliobacter (e.g., H. pylori), Klebsiella (K. pneumoniae), Legionella (e.g., L. pneumophila), Listeria (e.g., L. monocytogenes), Mycobacterium (e.g., M. avium, M. bovis, M. branderi, M. leprae, M. tuberculosis), Mycoplasma (e.g., M. genitalium, M. pneumoniae), Neisseria (e.g., N. gonorrheae, N. meningitidis), Pneumocystis (e.g., P. carinii), Pseudomonas (P. aeruginosa), Rickettsia, (e.g., R. rickettsia, R. typhi), Salmonella (e.g., S. enterica), Shigella (e.g., S. dysenteriae), Staphylococcus (e.g., S. aureus, S. epidermidis), Streptococcus (e.g., S. pneumoniae, S. pyogenes), Treponema (e.g., T. pallidum), Vibrio (e.g., V. cholerae, V. vulnificus), or Yersinia (e.g., Y. pestis). These include Gram-negative or Gram-positive bacteria, chlamydia, spirochetes, mycobacteria, and mycoplasmas.

The protozoan may be a species of the genus Cryptosporidium (e.g., C. hominis, C. parvum), Entamoeba (e.g., E. histolytica), Giardia (e.g., G. intestinalis, G. lamblia), Leishmania (e.g., L. amazonensis, L. braziliensi, L. donovani, L. mexicana, L. tropica), Plasmodium (e.g., P. falciparum, P. vivax), Toxoplasma (e.g., T. gondii), or Trypanosoma (e.g., T. bruci, T. cruzi).

The virus may be a DNA or RNA virus that infects humans and animals. DNA viruses include those belonging to the Adenoviridae, Iridoviridae, Papillomaviridae, Polyomavirididae, and Poxviridae families (Group I double-stranded DNA viruses); the Parvoviridae family (Group II single-stranded DNA viruses). RNA viruses include those belonging to the Birnaviridae and Reoviridae families (Group III double-stranded RNA viruses); the Arteriviridae, Astroviridae, Caliciviridae, Hepeviridae, and Roniviridae families (Group IV positive single-stranded RNA viruses); and the Arenaviridae, Bomaviridae, Bunyaviridae, Filoviridae, Paramyxoviridae, and Rhabdoviridae families (Group V negative single-stranded RNA viruses). Specifically-configured, double-stranded ribonucleic acid (dsRNA) is also known to treat infection by DNA viruses from the Herpesviridae family and RNA viruses from the Flaviviridae, Hepadnaviridae, Orthomyxoviridae, Picornaviridae, Retroviridae, and Togaviridae families; viruses of these families may or may not be included within the scope of the invention.

Cells of a subject undergoing abnormal proliferation may be a neoplasm or tumor (e.g., carcinoma, sarcoma, leukemia, lymphoma), especially a cell transformed by a tumor virus (e.g., DNA or RNA virus carrying a transforming gene or oncogene) or otherwise infected by a virus associated with cancer. For example, Epstein-Barr virus (EBV) is associated with nasopharyngeal cancer, Hodgkin's lymphoma, Burkitt's lymphoma, and other B-cell lymphomas; human hepatitis B and C viruses (HBV and HCV) are associated with liver cancer; human herpesvirus 8 (HHV8) is associated with Kaposi's sarcoma; human papillomaviruses (e.g., HPV6, HPV11, HPV16, HPV18, or combination thereof) are associated with cervical cancer, anal cancer, and genital warts; and human T-lymphotrophic virus (HTLV) is associated with T-cell leukemia or lymphoma. Cancers include those originating from the gastrointestinal (e.g., esophagus, colon, intestine, ileum, rectum, anus, liver, pancreas, stomach), genitourinary (e.g., bladder, kidney, prostate), musculoskeletal, nervous, pulmonary (e.g., lung), or reproductive (e.g., cervix, ovary, testicle) organ systems.

Poly(riboinosinic) is partially hybridized to poly(ribocytosinic₁₂uracilic) and can be represented as rIₙ·r(C₁₂U)ₙ. Other specifically-configured dsRNA that may be used are based on copolynucleotides selected from poly(CₙU) and poly(CₙG) in which n is an integer from 4 to 29 or are mismatched analogs of complexes of polyriboinosinic and polyribocytidilic acids, formed by modifying rIₙ·rCₙ to incorporate unpaired bases (uracil or guanine) along the polyribocytidylate (rCₙ) strand. Alternatively mismatched dsRNA may be derived from r(I)·r(C) dsRNA by modifying the ribosyl backbone of polyriboinosinic acid (rIₙ), e.g., by including 2′-O-methyl ribosyl residues. Mismatched dsRNA may be complexed with an RNA-stabilizing polymer such as lysine cellulose. Of these mismatched analogs of rIₙ·rCₙ, the preferred ones are of the general formula rIₙ·r(C₁₁₋₁₄U)ₙ and are described in U.S. Pat. Nos. 4,024,222 and 4,130,641; which are incorporated by reference. The dsRNA described therein generally are suitable for use according to the present invention. See also U.S. Pat. No. 5,258,369.

Specifically-configured dsRNA may be administered by any suitable local or systemic route including enteral (e.g., oral, feeding tube, enema), topical (e.g., patch acting epicutaneously, suppository acting in the rectum or vagina), and parenteral (e.g., transdermal patch; subcutaneous, intravenous, intramuscular, intradermal, or intraperitoneal injection; buccal, sublingual, or transmucosal; inhalation or instillation intranasally or intratracheally). The nucleic acid may be micronized for inhalation, dissolved in a vehicle (e.g., sterile buffered saline or water) for injection or instillation, or encapsulated in a liposome or other carrier for targeted delivery. Preferred are carriers that target the nucleic acid to the TLR3 receptor on antigen presenting cells and epithelium. For example, immature dendritic cells may be contacted in skin, mucosa, or lymphoid tissues. It will be appreciated that the preferred route may vary with condition and age of the subject, the nature of the infectious or neoplastic disease, and the chosen active ingredient.

The recommended dosage of the nucleic acid will depend on the clinical status of the subject and the experience of the physician or veterinarian in treating the viral infection or tumor burden. Specifically-configured dsRNA may be dosed at about 200 mg to about 400 mg by intravenous infusion to a 70 kg subject on a schedule of twice weekly, albeit the dose amount and/or frequency may be varied by the physician or veterinarian in response to the subject's condition. Cells or tissues that express TLR3 are preferred sites for delivering the nucleic acid, especially antigen presenting cells (e.g., dendritic cells and macrophages) and endothelium (e.g., respiratory and gastric systems). The effects of specifically-configured dsRNA may be inhibited or blocked by mutation of the TLR3 gene (e.g., deletion), down regulating its expression (e.g., siRNA), binding with a competitor for TLR3's ligand-binding site (e.g., neutralizing antibody) or a receptor antagonist, or interfering with a downstream component of the TLR3 signaling pathway (e.g., MyD88 or TRIF).

AMPLIGEN® poly(I:C₁₂U) provides a selective agent for dissecting out the effects of TLR3 activation on the immune system that was not previously available. Other agents like TLR adapters MyD88 and TRIF mediate signaling by all TLR or TLR3/TLR4, respectively. Thus, activation or inhibition of signaling through MyD88 or TRIF would not restrict the biological effects to those mediated by TLR3. Since the presence of TLR3 and its signaling is a requirement for AMPLIGEN® poly(I:C₁₂U) to act as a receptor agonist, one could assay for the absence of TLR3 mutations, the presence of TLR3 protein, intact TLR3-mediated signaling, or any combination thereof in the cell or tissue of a subject prior to administration of the agonist. Such confirmation of TLR3 activity can be performed before, during, or after administration of the agonist. The agonist can be used to restrict the immune response to activation of TLR3 without activating other Toll-like receptors or RNA helicases. For example, abnormal cytokine (e.g., IFN-α, IFN-β, IFN-γ, TNF-α, IL-6, IL-10, IL-12) production or co-stimulatory molecule (e.g., CD80, CD83, CD86) signaling may have resulted from at least infection by the microbe, abnormal cell proliferation, autoimmune damage, or neurodegeneration. This abnormality may be remodulated by using poly(I:C₁₂U) as a specific agonist of TLR3. Antigen presentation may be improved by conjugating the antigen (or a peptide analog thereof) to a ligand (or a receptor) that specifically binds to the cell surface (especially a component of the endosome-phagosome internalizing pathway) of one or more antigen presenting cells. The specific binding molecule may be an antibody to a cell surface molecule, or a derivative thereof (e.g., Fab, scFv).

### Examples

### Example 1

### Human Subjects

Human patients were enrolled with informed consent and were selected by the principal investigator as potential volunteers for additional analyses of serum cytokine levels and dendritic cell maturation marker expression. Patients could be newly enrolled or restarting poly(I:C₁₂U) treatment.

### Performance of Immune Panel

Our immune panel consists of measurements of cytokine serum levels (interferons, TNF-α, IL-6, IL-10, IL-12) and immune markers on blood cells (CD80, CD83, CD86). For patients consenting to such measurements, blood samples were collected from newly enrolled subjects and subjects restarting p(I:C₁₂U) infusion) prior to their initial 200 mg and subsequent 400 mg poly(I:C₁₂U) infusions as well as at 4±½, 24±2, and 72±2 hours post infusion. The specified collection times were selected to include points in the period between poly(I:C₁₂U) infusions. The immune panel was performed on these samples. For cytokine analyses, sera from the blood samples were frozen at −70° C. and shipped frozen from the study site to Hemispherx Biopharma, Inc. (New Brunswick, N.J.). Heparinized whole blood samples were also shipped from the study site overnight at ambient temperature for flow cytometric analyses of CD80, CD83, and CD86 by Celldex Therapeutics, Inc. (Phillipsburg, N.J.).

Interferons (IFN-α, IFN-β, IFN-γ) and inflammatory cytokines (TNF-α, IL-6, IL-10, IL-12p70) levels were measured using enzyme-linked immunosorbant assay (ELISA) kits according to manufacturers' instructions.

ELISA Kits

| Cytokine Assayed | Manufacturer | Lot Number |
|---|---|---|
| IFN-α | PBL Biomedical Laboratories | 3659 |
| IFN-β | BioSource | GL61204 |
| IFN-γ | BioSource | 1373742B |
| TNF-α | BioSource | 063704 |
| IL-6 | BioSource | 064004B |
| IL-10 | BioSource | 064405C |
| IL-12p70 | BioSource | 065004 |

Kits were from PBL Biomedical Laboratories, Piscataway, N.J. and BioSource International, Camarillo, Calif.

Celldex Therapeutics, Inc. (Phillipsburg, N.J.) was contracted for flow cytometric analyses of CD80, CD83, and CD86 expression. Following overnight shipment, blood samples were stained within one hour of receipt. Standard flow cytometry methods were employed for cell marker analyses and lysis of red blood cells. Dendritic cells were identified based on low level expression of monocyte, lymphocyte, and NK cell markers along with high HLA-DR expression. Dendritic cells were also characterized according to CD11c and CD123 expression. Monocytes were identified by side scatter analysis and expression of a monocyte lineage marker. Analyses of CD80, CD83, and CD86 expression were performed after cell type identification. Measure-ments from healthy volunteers served as controls and indicated normal distribution and levels of marker expression for mature DC such as CD80, CD83, and CD86.

### Results

Cytokine Levels: Results of cytokine analyses for each patient are presented in Tables 1 and 2. Zero was used as the value for results that had a negative absorbance value relative to a blank standard, or that were at or below the kit's detection limit (DL) as reported by the manufacturer. If the manufacturer did not specify a DL or indicated that the DL was less than a given value, all results were used. If the manufacturer provided expected normal cytokine ranges, they have been included for reference.

TABLE 1
Kinetics of Interferon Levels After Poly(I:C₁₂U) Infusion

| | Sample Time Relative to Infusion | | | |
|---|---|---|---|---|
| | | 4 hr Post- | 24 hr Post- | 72 hr Post- |
| Parameter/Patient | Pre-Infusion | Infusion | Infusion | Infusion |
| IFN-α (pg/mL) | | | | |
| NR: NP | | | | |
| JLC-109 | 741.27 | 756.93 | 749.80 | 733.13 |
| DMM-111 | 31.22 | 26.41 | 35.74 | 32.53 |
| LDM-010 | 182.43 | 166.97 | 175.00 | 170.38 |
| JOG-020 | 0 | 0 | 0 | 0 |
| Mean (SD) | 238 (344) | 237 (353) | 240 (348) | 234 (340) |
| IFN-β (IU/mL) | | | | |
| NR: NP | | | | |
| JLC-109 | 90.85 | 101.63 | 93.95 | 98.69 |
| DMM-111 | 0 | 0 | 0.16 | 0 |
| LDM-010 | 211.27 | 217.81 | 220.42 | 103.95 |
| JOG-020 | 0 | 0 | 0 | 0 |
| Mean (SD) | 75 (100) | 79 (103) | 78 (104) | 50 (58) |
| IFN-γ (pg/mL) | | | | |
| NR: 0-5 pg/mL | | | | |
| JLC-109 | 56.44 | 53.70 | 68.29 | 56.71 |
| DMM-111 | 22.46 | 111.551 | 17.26 | 14.79 |
| LDM-010 | 0 | 0 | 0 | 0 |
| JOG-020 | 449.52 | 415.82 | 466.16 | 462.60 |
| Mean (SD) | 132 (212) | 145 (186) | 137 (220) | 133 (220) |

NR, normal range; NP, not provided by kit manufacturer

TABLE 2
Kinetics of Cytokine Levels After Poly(I:C₁₂U) Infusion

| | Sample Time Relative to Infusion | | | |
|---|---|---|---|---|
| | | 4 hr Post- | 24 hr Post- | 72 hr Post- |
| Parameter/Patient | Pre-Infusion | Infusion | Infusion | Infusion |
| TNF-α (pg/mL) | | | | |
| NR: 0-20 pg/mL | | | | |
| JLC-109 | 7.31 | 11.19 | 11.87 | 12.56 |
| DMM-111 | 5.02 | 17.12 | 14.61 | 9.36 |
| LDM-010 | 7.08 | 7.99 | 8.68 | 9.36 |
| JOG-020 | 8.45 | 8.68 | 10.50 | 11.42 |
| Mean (SD) | 7.0 (1.4) | 11.3 (4.2) | 11.4 (2.5) | 10.7 (1.6) |
| IL-6 (pg/mL) | | | | |
| NR: NP | | | | |
| JLC-109 | 72.19 | 80.14 | 72.00 | 67.35 |
| DMM-111 | 0 | 63.66 | 0.10 | 0 |
| LDM-010 | 1.36 | 2.03 | 2.71 | 5.81 |
| JOG-020 | 30.04 | 10.27 | 17.73 | 14.44 |
| Mean (SD) | 26 (34) | 39 (39) | 23 (33) | 22 (31) |
| IL-10 (pg/mL) | | | | |
| NR: <1 pg/mL | | | | |
| JLC-109 | 0 | 1.40 | 0 | 0 |
| DMM-111 | 1.05 | 0.77 | 0.49 | 3.43 |
| LDM-010 | 0 | 0 | 0.49 | 0 |
| JOG-020 | 0 | 0 | 0.49 | 0.49 |
| Mean (SD) | 0.26 (0.53) | 0.54 (0.68) | 0.37 (0.25) | 0.98 (1.65) |
| IL-12p70 (pg/mL) | | | | |
| NR: <0.79 pg/mL | | | | |
| JLC-109 | 6.59 | 7.47 | 8.70 | 7.54 |
| DMM-111 | 16.57 | 20.13 | 25.17 | 16.20 |
| LDM-010 | 5.57 | 8.48 | 12.20 | 14.37 |
| JOG-020 | 32.79 | 43.31 | 38.24 | 46.74 |
| Mean (SD) | 15 (13) | 20 (17) | 21 (13) | 21 (17) |

NR, normal range; NP, not provided by kit manufacturer

Mean values and standard deviations (SD) were calculated. But interpretation of the mean values is limited due to the small number of patients and the data variability. Baseline cytokine levels varied widely from patient to patient. This is not surprising as inconsistency in indicators of immune system activation is characteristic of patients diagnosed with chronic fatigue syndrome (CFS), and has made it difficult to develop diagnostic tests difficult. In order to facilitate data interpretation, cytokine data are presented on a per patient basis. A descriptive discussion of the data is presented. Statistical analyses were not performed.

Three of four patients had elevated pre-infusion levels of IFN-α and IFN-γ, and two of four had high pre-infusion levels of IFN-β. Considering changes in IFN-α levels, one patient (LDM-010) had levels lower than pre-infusion at all subsequent time points, one patient (JOG-020) had undetectable levels at all time points, and two patients increased for at least one post-infusion time point. Maximum increases in IFN-α were 2% to 15% above pre-infusion levels. The same patient who had undetectable levels of IFN-α at all time points (JOG-020) also had undetectable levels of IFN-β. All other patients had increased IFN-β levels for at least one time point, although the increase for one patient (DMM-111) was substantially lower (about 0.1%) than the levels measured for other patients. Maximum increases in IFN-β for the other two patients ranged from 4.3% to 12% above pre-infusion levels. All patients except one demonstrated increases in IFN-γ for at least one post-infusion time point; the fourth patient (LDM-010) had undetectable levels at all time points. For two of the patients, maximum increases in IFN-γ ranged from 3.7% and 21% above pre-infusion levels. One patient (DMM-111) demonstrated a 397% increase over the pre-infusion level at 4 hours post-infusion, but subsequent measurements were below the pre-infusion baseline. All patients had levels of IFN-α, IFN-β, and IFN-γ at or below pre-infusion levels by 72 hours after infusion.

All patients had normal pre-infusion levels of TNF-α based on the expected range for healthy individuals from 0 to 20 pg/mL. Relative to pre-infusion levels, all patients demonstrated small increases in TNF-α at each post-infusion time point, although levels for one patient (DMM-111) decreased at the 72 hour time point relative to the 4 and 24 hour points, but all patients remained in the normal range. Maximum TNF-α increases ranged from 32% to 241% above pre-infusion levels. As with TNF-α, mean results for IL-10 at each time point fell below the expected normal concentration. Two patients (LDM-101, JOG-020) demonstrated elevations within the expected range, and two (JLC-109, DMM-111) had increases above the expected concentration. Only one patient (DMM-111) had an IL-10 level above the expected concentration at 72 hours; the IL-10 level for this patient at 72 hours was greater than 3 fold higher than pre-infusion levels. All patients had elevated pre-infusion levels of IL-12p70 (expected range up to 0.79 pg/mL), and all but one demonstrated sustained increases over pre-infusion levels for all time points. The single patient who did not have a sustained IL-12p70 increase (DM-111) decreased to 2.2% below pre-infusion levels at 72 hours. Maximum IL-12p70 increases ranged from 32% to 158% above pre-infusion levels.

Three of four patients had elevated pre-infusion levels of IL-6. One patient (JOG-020) had decreased IL-6 levels relative to pre-infusion at all subsequent time points. For the one patient (DMM-111) with undetectable IL-6 pre-infusion, levels increased to high levels at 4 hours post-infusion then returned to baseline. The same patient demonstrated peaks in IFN-γ and TNF-α at 4 hours post-infusion. Only one patient (LDM-010) had levels of IL-6 higher than pre-infusion levels at 72 hours. Only one patient (DMM-111) had a detectable IL-10 level pre-infusion, and the level was not greatly elevated over the expected concentration of less than 1 pg/mL.

Results of cytokine analyses indicate elevated pre-infusion levels of IL-12p70 and modest increases for the 72 hours following poly(I:C₁₂U) infusion. However, there were no apparent differences in IFN-α, IFN-β, and IFN-γ levels over the monitored post infusion period, mean TNF-α and IL-10 levels were within the expected normal ranges at each time point, and IL-6 responses had decreased to pre-infusion levels for three of four patients by 72 hours post-infusion. In conclusion, no substantial patterns of modulation of cytokine levels including pro-inflammatory cytokines TNF-α, IL-6 and IL-12 were documented.

### Dendritic Cell Maturation Markers

Results of DC maturation marker analyses are reported as percentage of positive-staining cells and by expression level (mean fluorescence intensity, MFI) and are presented as mean (SD) unless otherwise indicated. Data for healthy volunteers not treated with poly(I:C₁₂U) are reported for comparison. Due to the large variability in CD80, CD83, and CD86 data, tables detailing results on a per individual basis are included. Statistical analyses were not performed.

Mean (SD) percentages of CD123+ DC, CD11+ DC, and monocytes are presented in Table 3. Values for healthy volunteers were included as normal values (see Table 4). Statistical analyses were not performed. Healthy volunteers did not receive poly(I:C₁₂U) infusion. Values for CFS patients are reported for specified time points relative to poly(I:C₁₂U) infusion. Mean values were calculated over all measurements for all patients at each time point.

TABLE 3
Poly(I:C₁₂U) Effects on Cell Populations

| | Number of Cells (% of Leukocytes) | | |
|---|---|---|---|
| | CD123⁺ | CD11⁺ | Monocytes |
| Healthy Volunteers^{a} | 0.17 (0.06) | 0.27 (0.11) | 6.10 (1.12) |
| (n = 6) | | | |
| CFS Patients (n = 4) | | | |
| Pre-infusion | 0.15 (0.09) | 0.12 (0.04) | 4.77 (0.77) |
| 4 hr Post-Infusion | 0.07 (0.03) | 0.17 (0.13) | 4.40 (1.10) |
| 24 hr Post-Infusion | 0.12 (0.03) | 0.08 (0.03) | 3.01 (0.89) |
| 72 hr Post-Infusion | 0.13 (0.05) | 0.19 (0.06) | 4.95 (0.48) |

^{a}Healthy volunteers received no poly(I:C₁₂U) infusions

TABLE 4
Individual Results for Dendritic Cell
Type Percentages in Healthy Volunteers

| Healthy Volunteer | CD123⁺ | CD11c⁺ | Monocytes |
|---|---|---|---|
| 1 | 0.21 | 0.33 | 4.93 |
| | 0.23 | 0.32 | 4.87 |
| | 0.22 | 0.35 | 4.93 |
| | 0.2 | 0.31 | 4.58 |
| Mean | 0.22 | 0.33 | 4.83 |
| SD | 0.01 | 0.02 | 0.17 |
| 2 | 0.14 | 0.19 | 5.02 |
| | 0.14 | 0.16 | 4.92 |
| | 0.15 | 0.16 | 4.82 |
| | 0.15 | 0.15 | 5.01 |
| Mean | 0.15 | 0.17 | 4.94 |
| SD | 0.01 | 0.02 | 0.09 |
| 3 | 0.12 | 0.22 | 5.72 |
| | 0.13 | 0.21 | 5.3 |
| | 0.12 | 0.19 | 5.3 |
| | 0.13 | 0.22 | 5.52 |
| Mean | 0.13 | 0.21 | 5.46 |
| SD | 0.01 | 0.01 | 0.20 |
| 4 | 0.08 | 0.2 | 6.27 |
| | 0.08 | 0.19 | 6.62 |
| | 0.09 | 0.23 | 6.67 |
| | 0.09 | 0.19 | 6.87 |
| Mean | 0.09 | 0.20 | 6.61 |
| SD | 0.01 | 0.02 | 0.25 |
| 5 | 0.19 | 0.23 | 7.53 |
| | 0.18 | 0.24 | 7.71 |
| | 0.18 | 0.23 | 7.67 |
| | 0.18 | 0.23 | 7.67 |
| Mean | 0.18 | 0.23 | 7.65 |
| SD | 0.01 | 0.00 | 0.08 |
| 6 | 0.26 | 0.49 | 7.26 |
| | 0.23 | 0.5 | 7.02 |
| | 0.24 | 0.49 | 7.11 |
| | 0.26 | 0.49 | 7.14 |
| Mean | 0.25 | 0.49 | 7.13 |
| SD | 0.02 | 0.01 | 0.10 |

Pre-infusion values for CFS patients were comparable with healthy volunteers' levels; the percentage of CD11+ cells was at the low end of the range for healthy volunteers as defined by the mean and SD. Mean values were below those measured for healthy volunteers for CD123+ cells 4 hours post-infusion and for CD11+ cells and monocytes 24 hours post-infusion. Due to the small population sample, changes experienced by one patient could noticeably affect results. For example, patient DMM-111 experienced a 10-fold drop in percentage of CD123+ cells from pre-infusion to 4 hours post-infusion (see Table 5). One consistent change was a decrease in the percentage of monocytes demonstrated by CFS patients 24 hours post-infusion. Monocyte numbers recovered by 72 hours post infusion (see Table 5). Overall, percentages of CD123+ cells, CD11+ cells, and monocytes (mono) were slightly low, but not out of the range of values for healthy volunteers.

In general, treatment with poly(I:C₁₂U) decreased the percentage of cells expressing the mature DC markers CD80, CD83, and CD86, and it increased their expression levels. CFS patients tended to start with more positive cells having lower expression levels than healthy volunteers who received no poly(I:C₁₂U). Thus, the ability of poly(I:C₁₂U) to decrease the number of positive cells and to increase the expression level of DC maturation markers normalized CFS patients' profiles such that they more closely resembled those of healthy volunteers.

TABLE 5
Individual Results for Dendritic Cell Type Percentages in CFS Patients

| | Day 1 Pre-Infusion | | | Day 1 Post-Infusion | | | 24 Hours Post-Infusion | | | 72 Hours Post-Infusion | | |
|---|---|---|---|---|---|---|---|---|---|---|---|---|
| Patient ID | CD123+ | CD11c+ | mono | CD123+ | CD11c+ | mono | CD123+ | CD11c+ | mono | CD123+ | CD11c+ | mono |
| LDM-010 | 0.11 | 0.07 | 3.81 | 0.07 | 0.18 | 3.87 | 0.08 | 0.12 | 2.3 | 0.13 | 0.27 | 5.69 |
| | 0.14 | 0.08 | 4.82 | 0.06 | 0.21 | 3.22 | 0.08 | 0.14 | 2.12 | 0.11 | 0.29 | 5.56 |
| | 0.11 | 0.1 | 3.2 | 0.07 | 0.21 | 3.37 | 0.1 | 0.11 | 2.06 | 0.13 | 0.27 | 5.72 |
| | 0.13 | 0.08 | 3.75 | 0.08 | 0.23 | 3.21 | 0.11 | 0.09 | 2.39 | 0.12 | 0.28 | 5.47 |
| Mean | 0.12 | 0.08 | 3.90 | 0.07 | 0.21 | 3.42 | 0.09 | 0.12 | 2.22 | 0.12 | 0.28 | 5.61 |
| SD | 0.02 | 0.01 | 0.68 | 0.01 | 0.02 | 0.31 | 0.02 | 0.02 | 0.15 | 0.01 | 0.01 | 0.12 |
| JOG-020 | 0.09 | 0.11 | 6.35 | 0.08 | 0.1 | 4.84 | 0.08 | 0.07 | 3.25 | 0.08 | 0.13 | 4.85 |
| | 0.1 | 0.11 | 4.75 | 0.08 | 0.11 | 4.78 | 0.08 | 0.08 | 3.49 | 0.09 | 0.14 | 4.94 |
| | 0.12 | 0.13 | 4.96 | 0.1 | 0.1 | 4.69 | 0.08 | 0.07 | 3.09 | 0.08 | 0.15 | 4.97 |
| | 0.11 | | 4.28 | 0.1 | 0.11 | 4.69 | 0.09 | 0.08 | 3.04 | 0.1 | 0.12 | 4.75 |
| Mean | 0.11 | 0.12 | 5.09 | 0.09 | 0.11 | 4.75 | 0.08 | 0.08 | 3.22 | 0.09 | 0.14 | 4.88 |
| SD | 0.01 | 0.01 | 0.89 | 0.01 | 0.01 | 0.07 | 0.01 | 0.01 | 0.20 | 0.01 | 0.01 | 0.10 |
| JLC-109 | 0.09 | 0.11 | 5 | 0.13 | 0.41 | 6.63 | 0.13 | 0.06 | 5.45 | 0.1 | 0.16 | 4.43 |
| | 0.06 | 0.12 | 4.68 | 0.12 | 0.35 | 4.92 | 0.13 | 0.08 | 3.66 | 0.1 | 0.17 | 4.36 |
| | 0.09 | 0.13 | 4.56 | 0.06 | 0.41 | 5.77 | 0.15 | 0.14 | 2.21 | 0.12 | 0.18 | 4.37 |
| | 0.09 | 0.13 | 4.53 | 0.1 | 0.27 | 6.2 | 0.14 | 0.09 | 1.8 | 0.11 | 0.17 | 4.21 |
| Mean | 0.08 | 0.12 | 4.69 | 0.10 | 0.36 | 5.88 | 0.14 | 0.09 | 3.28 | 0.11 | 0.17 | 4.34 |
| SD | 0.02 | 0.01 | 0.22 | 0.03 | 0.07 | 0.73 | 0.01 | 0.03 | 1.65 | 0.01 | 0.01 | 0.09 |
| DMM-111 | 0.33 | 0.1 | 5.45 | 0.03 | 0.02 | 3.94 | 0.13 | 0.04 | 3.44 | 0.2 | 0.18 | 4.82 |
| | 0.33 | 0.11 | 5.26 | 0.03 | 0.02 | 3.44 | 0.14 | 0.03 | 3.26 | 0.21 | 0.17 | 4.98 |
| | 0.32 | 0.22 | 5.48 | 0.03 | 0.02 | 3.39 | 0.16 | 0.05 | 3.6 | 0.21 | 0.16 | 4.85 |
| | 0.24 | 0.16 | 5.42 | 0.03 | 0.02 | 3.49 | 0.16 | 0.04 | 2.92 | 0.22 | 0.2 | 5.29 |
| Mean | 0.31 | 0.15 | 5.40 | 0.03 | 0.02 | 3.57 | 0.15 | 0.04 | 3.31 | 0.21 | 0.18 | 4.99 |
| SD | 0.04 | 0.06 | 0.10 | 0.00 | 0.00 | 0.25 | 0.01 | 0.01 | 0.29 | 0.01 | 0.02 | 0.21 |

Individual patient results tended to reflect the mean changes shown in Tables 6 to 8 with decreases in the proportions of positive cells and increased expression levels at the 24 hr and 72 hr post-infusion time points. There were some exceptions to this pattern. For example, percentages of CD80 and CD86 expressing cells did not decrease as noticeably among CD11+ cells as among CD123+ cells. In addition, monocytes from CFS patients were similar to those from healthy volunteers in terms of percentages of cells expressing CD86 and in CD86 expression level.

TABLE 6
Kinetics of Maturation Marker Expression in CD123+ Dendritic Cells After Poly(I:C₁₂U) Infusion

| | CD80 | | CD83 | | CD86 | |
|---|---|---|---|---|---|---|
| | Positive | | Positive | | Positive | |
| | Cells (%) | MFI^{b} | Cells (%) | MFI | Cells (%) | MFI |
| Healthy Volunteers^{a} | 0.8 (1.7) | 55.0 (33.2) | 11.1 (7.7) | 78.6 (69.0) | 35.0 (17.5) | 71.8 (25.1) |
| (n = 6) | | | | | | |
| CFS Patients (n = 4) | | | | | | |
| Pre-infusion | 5.0 (8.0) | 16.9 (8.8) | 38.5 (24.2) | 20.8 (6.5) | 59.3 (31.7) | 33.6 (9.6) |
| 4 h Post-infusion | 1.8 (2.2) | 18.5 (8.2) | 51.5 (20.2) | 21.5 (7.4) | 63.8 (7.7) | 27.8 (10.0) |
| 24 h Post-infusion | 2.3 (2.4) | 55.3 (50.1) | 8.1 (2.8) | 68.5 (55.9) | 24.8 (7.9) | 74.3 (62.6) |
| 72 h Post-infusion | 0.3 (1.6) | 68.5 (48.1) | 5.1 (2.3) | 92.7 (10.4) | 23.1 (4.6) | 99.3 (17.2) |

^{a}Healthy volunteers received no poly(I:C₁₂U);
^{b}MFI, mean fluorescence intensity

TABLE 7
Kinetics of Maturation Marker Expression in CD11+
Dendritic Cells After Poly(I:C₁₂U) Infusion

| | CD80 | | CD83 | | CD86 | |
|---|---|---|---|---|---|---|
| | Positive | | Positive | | Positive | |
| | Cells (%) | MFI^{b} | Cells (%) | MFI | Cells (%) | MFI |
| Healthy Volunteers^{a} | 1.1 (0.6) | 111.1 (145.8) | 7.8 (6.3) | 65.1 (40.1) | 87.0 (9.4) | 98.9 (19.6) |
| (n = 6) | | | | | | |
| CFS Patients (n = 4) | | | | | | |
| Pre-infusion | 0.4 (2.0) | 23.5 (10.7) | 23.4 (6.7) | 21.3 (9.0) | 97.1 (1.8) | 67.4 (9.4) |
| 4 h Post-infusion | 3.5 (4.7) | 17.4 (9.4) | 19.7 (8.8) | 18.6 (10.2) | 97.4 (1.6) | 66.6 (19.8) |
| 24 h Post-infusion | 3.9 (6.7) | 46.1 (42.1) | 10.4 (8.0) | 81.4 (59.7) | 67.0 (31.5) | 98.8 (80.3) |
| 72 h Post-infusion | −0.1 (0.5) | 115.3 (102.5) | 2.5 (1.4) | 98.5 (34.3) | 82.9 (11.4) | 101.4 (17.6) |

^{a}Healthy volunteers received no poly(I:C₁₂U);
^{b}MFI, mean fluorescence intensity

TABLE 8
Kinetics of Maturation Marker Expression
in Monocytes After Poly(I:C₁₂U) Infusion

| | CD80 | | CD83 | | CD86 | |
|---|---|---|---|---|---|---|
| | Positive | | Positive | | Positive | |
| | Cells (%) | MFI^{b} | Cells (%) | MFI | Cells (%) | MFI |
| Healthy Volunteers^{a} | 1.3 (0.9) | 86.6 (30.6) | 11.6 (8.0) | 80.9 (31.6) | 66.0 (27.4) | 119.7 (30.8) |
| (n = 6) | | | | | | |
| CFS Patients (n = 4) | | | | | | |
| Pre-infusion | 2.6 (4.0) | 52.3 (13.2) | 26.9 (6.3) | 56.4 (11.4) | 86.8 (5.9) | 109.3 (12.6) |
| 4 h Post-infusion | 1.7 (1.8) | 42.5 (12.0) | 34.5 (17.3) | 47.9 (13.7) | 92.0 (2.7) | 89.8 (15.5) |
| 24 h Post-infusion | 0.9 (1.5) | 61.4 (33.4) | 19.5 (5.9) | 80.9 (43.0) | 83.2 (8.5) | 172.2 (62.0) |
| 72 h Post-infusion | 0.0 (0.4) | 76.1 (19.2) | 9.9 (2.5) | 82.5 (20.3) | 86.7 (3.6) | 143.9 (28.9) |

^{a}Healthy volunteers received no poly(I:C₁₂U);
^{b}MFI, mean fluorescence intensity

In summary, poly(I:C₁₂U) infusion did not dramatically affect the numbers of CD123+ DC, CD11+ DC, or monocytes. Following poly(I:C₁₂U) treatment, CFS patients experienced normalization in the percentages of DC expressing maturation markers and in CD maturation marker expression levels. These trends, particularly the increase in CD maturation markers, were consistently observed in all four patients, revealing a distinct pattern not recognized in cytokine level modulation.

### Example 2

### DsRNA Induces Durable and Protective Adaptive Immunity

CD4+ Th1-type immunity is implicated in resistance to global infectious diseases. To improve the efficacy of T cell immunity induced by HIV vaccines, a protein-based approach was developed that directly harnesses the function of dendritic cells (DC) in intact lymphoid tissues. Antigenic proteins are selectively delivered to dendritic cells by antibodies targeted to DEC-205, a receptor for antigen presentation. DsRNAs independently serve as adjuvants to allow a DC-targeted protein to induce protective CD4+ T cell responses at a mucosal surface (i.e., the airway). Following two doses of DEC-targeted, HIV gag p24 along with dsRNA, the immune CD4+ T cells have qualitative features that are correlated with protective function. The T cells simultaneously produce IFN-γ, TNF-α, and IL-2 in high amounts and for prolonged times. The T cells also proliferate and continue to secrete IFN-γ in response to HIV gag p24. The adjuvant role of poly(I:C) requires TLR3 and MDA5 receptors, but the analogous poly(I:C₁₂U) requires TLR3 only (see results below). Both poly(I:C) and poly(I:C₁₂U) are safe adjuvants when used with DC-targeted vaccines to induce abundant CD4+ Th1 cells with features like multifunctionality and proliferative capacity.

T cell mediated immunity is implicated in the resistance to global infectious diseases like HIV, malaria, and tuberculosis. A critical component is the CD4+ Th1 helper cell, which can produce large amounts of IFN-γ and TNF-α, exert cytolytic activity on MHC class II+ targets, and sustain functional CD8+ T memory cells.

Dendritic cells are antigen presenting cells that induce strong T cell-based responses. For example, when a subset of dendritic cells that express the endocytic receptor DEC-205 (“DEC”) is loaded with antigen ex vivo and reinfused into mice, the dendritic cells expand antigen-specific helper T cells to primarily produce IFN-γ. In vivo, DEC+ dendritic cells mediate antigen presentation on both MHC class I and II products, leading to clonal expansion of killer and T helper cells respectively. To better harness DC biology in vaccine design, we have been developing an approach that targets antigens directly to the endocytic receptor DEC-205.

Along with uptake of antigen, dendritic cells must differentiate or mature to immunize to foreign antigens. This maturation can be achieved with adjuvants, including chemically defined ligands for pattern recognition receptors, such as the Toll-like receptors (TLRs). The type of TLR ligand influences the outcome of the immune response. With respect to antigen-specific, CD4+ and CD8+ immunity in mice and monkeys, TLR ligands including conjugates of TLR7/8 ligand to HIV gag p41 serve as active adjuvants.

Ligands for pattern recognition receptors have not been evaluated as potential safe adjuvants for T cell based protective immunity with DC-targeted HIV vaccines. dsRNA was introduced alone as an adjuvant to show that it adjuvants a DC-targeted vaccine to induce CD4+ T cell immunity that is quantitatively and qualitatively robust by current criteria, and also protective in a lung infection model.

The development of immunity to foreign proteins requires the coadministration of adjuvant. DsRNA is shown to be a superior adjuvant for inducing a strong CD4+ T cell response to α-DEC-HIV gag p24: i.e., the frequency of IFN-γ secreting T cells corresponded to 0.2-6% of total CD3+ CD4+ T cells. But poly(I:C) was needed during both priming and booster doses of vaccine.

A long felt need in the art is to define criteria for high quality protective T cells during natural infection or vaccination. To assess the quality of CD4+ T cells induced by using dsRNA as adjuvant, α-DEC-p24 and graded doses of poly(I:C) were given over six weeks. One group received α-DEC-p24 and poly(I:C) only at the boost. Two weeks after the boost, the frequency of gag-specific, CD4+ T cells producing IFN-γ, TNF-α, or IL-2 was greatest with two doses of α-DEC-p24 mAb and 50 μg poly(I:C).

The capacity of individual gag-specific T cells to secrete multiple cytokines was examined. Such multifunctional T cells contribute more effectively to protective immunity to select infectious pathogens including HIV. Two weeks after prime-boost immunization with α-DEC-p24 and 50 μg poly(I:C), roughly 50% of the gag specific CD4+ T cells produce all three cytokines: IFN-γ, TNF-α, and IL-2. Total frequencies of cytokine producers were less with two doses of α-DEC-p24 and 10 μg poly(I:C), or a single dose of α-DEC-p24 and 50 μg poly(I:C). The amount of each cytokine (median fluorescence intensity or MFI) made by gag-responsive cells was assessed because this parameter is an important correlate for protective CD4+ immunity in the L. major model. The MFI of cells producing three cytokines (i.e., IFN-γ, TNF-α, and IL-2) was higher than the MFI of cells producing two or only one cytokine. Therefore, the effector CD4+ T cells induced with α-DEC-p24 and dsRNA have features which are currently associated with superior Th1 immunity, such as polyfunctionality and high cytokine production.

To assess the persistence of HIV gag-specific, cytokine-producing (“effector”) CD4+ T cells after prime-boost immunization with α-DEC-p24 plus poly(I:C), analyses were performed two and seven weeks after the boost. When data is summarized for three experiments each in BALB/c and C57BL/6 mice, adaptive effector CD4+ T cells were shown to persist for at least seven weeks. Interestingly, the percentage of cells producing all three cytokines (i.e., IFN-γ, TNF-α, and IL-2) remained stable from week 2 to week 7 after the boost. Therefore, following prime-boost immunization adjuvanted with dsRNA, CD4+ effector T cells persisted for several weeks.

Recent findings indicate that the capacity of CD4+ Th1 cells to proliferate and to produce IFN-γ against HIV-1 strongly associates with low HIV-1 RNA and proviral DNA loads. Thus, whether these T cell features could be induced by a DC-targeted vaccine was assessed. Two weeks after the boost, CD4+ T cells responded to gag p24 specifically by proliferating and producing IFN-γ. There was no response to the negative control (i.e., mixture of gag p17 peptides). Fewer responding CD4+ T cells were seen with two doses of α-DEC-p24 plus 10 μg or 2 μg poly(I:C), or one dose of α-DEC-p24 plus 50 μg poly(I:C). Therefore, prime-boost immunization with α-DEC-p24 and dsRNA induces proliferating CD4+ T cells.

Persistence of proliferative, HIV gag-specific CD4+ T cells after prime-boost immunization with α-DEC-p24 and poly(I:C) was then assessed. The results from three experiments each in BALB/c and C57BL/6 mice indicated that such CD4+ T cells persisted at least seven weeks in the spleen. Therefore, a prime boost with α-DEC-p24 and 50 μg poly(I:C) induces long-lived proliferating, IFN-γ secreting CD4+ T cells.

Furthermore, poly(I:C) also serves as an adjuvant for CD4+ T cell response to α-DEC-nef. Proliferating, IFN-γ secreting CD4+ T cells were elicited by immunization. They indicate that a good quantity and quality of CD4+ T cells will respond to HIV antigens, both nef and gag, when dsRNA is used as adjuvant.

To determine if dsRNA could generate long lasting protective immunity at a mucosal surface, BALB/c and C57BL/6 mice were vaccinated and then challenged intranasally with recombinant vaccinia-gag virus six to eight weeks after prime-boost immunization with α-DEC-gag and poly(I:C). In initial protection experiments, a dose of 50 μg poly(I:C) gave optimal protection and reduced virus titers in the lung. More detailed studies with this dose of poly(I:C) were carried out. The results from three experiments each in BALB/c and C57BL/6 mice showed that mice injected with the negative control (i.e., PBS) lost weight and developed high titers of virus in the lung (>10⁸ PFU/ml) over six to seven days. By both criteria, reduced weight loss and virus growth, two doses of α-DEC-p24 and 50 μg poly(I:C) provided better protection relative to two doses of control Ig-p24 or one dose of α-DEC-p24 plus poly(I:C). Therefore, prime-boost immunization with α-DEC-gag and dsRNA elicits protection at a mucosal surface.

When CD4+ cells were depleted from vaccinated mice before the challenge with vaccinia-gag, it was verified by vaccinia challenge that CD4+ T cells contributed to protection. Challenge experiments with DEC−/− null mice were also performed. A lack of protection was observed, showing that DEC was essential to develop DC-targeted protective immunity. TLR3−/− null mice, however, were fully protected against vaccinia-gag challenge after vaccination with two doses of α-DEC-p41 and poly(I:C). Therefore, although TLR3 was not required for protection when poly(I:C) was used as adjuvant, TLR but was essential for the action of poly(I:C₁₂U).

Poly(I:C₁₂U), an analog of poly(I:C), was also evaluated for adjuvant activity because poly(I:C₁₂U) exhibits minimal toxicity even at high doses but shares some immunomodulatory properties with poly(I:C). C×B6 F1 mice were injected with two doses of α-DEC-p24 plus increasing doses of poly(I:C) or poly(I:C₁₂U). Proliferation of CD3+CD4+ T cells in response to HIV gag p24 peptides was measured one week after boosting. Both forms of dsRNA induced CD4+ T cell responses that were dose-dependent and antigen specific.

To determine the pattern recognition receptors required for adjuvant activity using poly(I:C) and poly(I:C₁₂U), mice of wild-type, TLR3−/− null, or MDA5−/− null genetic background were compared. Poly(I:C) showed some adjuvant activity in TLR3−/− null mice, whereas poly(I:C₁₂U) surprisingly could not elicit CD4+ IFN-γ secreting T cells in the same genetic background. Both adjuvants elicited responses in MDA5−/− null mice. Taken together, the data indicate that TLR3 is essential for the adjuvant role of poly(I:C₁₂U), but poly(I:C) may be able to utilize both the cell-surface receptor TLR3 and the cytosolic sensor MDA5.

Dendritic cells are potent inducers of T cell-mediated immunity. Therefore, they are attractive targets when improvement in vaccine efficacy is sought. As an example, when antigenic proteins are selectively delivered through conjugates with antibodies targeting them to APC-specific surface molecules, antigen presentation and immune responses develop with much greater efficacy relative to nontargeted antigen. One receptor used here is endocytic receptor DEC-205 or CD205, which is expressed on dendritic cells in the T cell areas. dsRNA was used alone as a DC maturation stimulus and several features of the quality of CD4+ T cell immunity were studied, including memory. Our data reveal the potential of dsRNA to serve as an adjuvant for a prime-boost, protein vaccine, inducing long-lived and protective Th1 CD4+ T cells of superior quality and quantity.

The quality of the CD4+ T cell response with dsRNA as an adjuvant is shown first by its polyfunctionality, i.e., the T cells produced multiple cytokines such as IFN-γ, TNF-α, and IL-2, and in high amounts. It was also found that DEC-targeted vaccine induced high frequencies of IFN-γ-producing and proliferating CD4+ T cells, a feature of T cell immunity that has not been demonstrated in the prior art by other vaccine approaches. Increased frequencies of proliferating and multifunctional CD4+ T cells are currently regarded to be valuable features of Th1 immunity and are associated with better control of HIV and better protection in the L. major model.

Another interesting result of using DC-targeted HIV gag p24 was the induction of long-lasting protective CD4+ T cell immunity to vaccinia-gag in a DEC dependent manner. A contribution of CD4+ T cells to protection against vaccinia was detected. Given the critical role of IFN-γ in resistance to infection, high levels of IFN-γ as well as lysis by infected MHC class II+ targets by CD+ Th1 helper cells may both contribute to resistance induced by DEC targeted proteins together with dsRNA.

poly(I:C) can be recognized by both TLR3 endosomal and MDA5 cytosolic receptors. TLR3 and MDA5 were both found to contribute to adjuvant action and protective immunity with poly(I:C). In contrast, TLR3 is exclusively needed for the adjuvant role of poly(I:C₁₂U). Additionally, dsRNA induces type I interferons, which promotes cross-presentation by dendritic cells and survival of CD8+ T cells.

Although dsRNA has been used as adjuvant to enhance the immunogenicity to a vaccine protein in mice, its ability to induce CD4+ T cell responses that are also protective has not been demonstrated previously. Targeting of dsRNA-adjuvanted vaccine protein to dendritic cells and their endocytic receptor DEC-205 should favor the development of the particular kind of Th1 CD4+ T cell immunity that is implicated in resistance to several global infectious diseases.

Patents, patent applications, books, and other publications cited herein are incorporated by reference in their entirety.

In stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity which a person skilled in the art would understand does not affect operation of the invention or its patentability.

All modifications and substitutions that come within the meaning of the claims and the range of their legal equivalents are to be embraced within their scope. A claim which recites “comprising” allows the inclusion of other elements to be within the scope of the claim; the invention is also described by such claims reciting the transitional phrases “consisting essentially of” (i.e., allowing the inclusion of other elements to be within the scope of the claim if they do not materially affect operation of the invention) or “consisting of” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A method of initiating an innate immune response mediated only by Toll-Like Receptor 3 (TLR3), said method comprising administration to a subject of at least poly(I:C₁₁₋₁₄U) in an amount sufficient to activate TLR3 without activating other Toll-like receptors or RNA helicases, or without inducing an excessive amount of one or more pro-inflammatory cytokines.

2. The method according to claim 1, wherein at least cytokine production or co-stimulatory molecule signaling which had been initiated by autoimmune damage or neurodegeneration in the subject is remodulated by poly(I:C₁₁₋₁₄U).

3. A method of treating a subject infected with a microbe, said method comprising administration of a pharmaceutical composition comprised of poly(I:C₁₁₋₁₄U) in an amount sufficient to bind to Toll-Like Receptor 3 (TLR3) and to reduce or eliminate infection of the subject by the microbe.

4. The method according to claim 3, wherein the subject is infected by a microbe selected from the group consisting of bacteria, protozoa, and viruses.

5. A method of treating a subject bearing a tumor or other transformed cell, said method comprising administration of a pharmaceutical composition comprised of poly(I:₁₁₋₁₄U) in an amount sufficient to bind to Toll-Like Receptor 3 (TLR3) and to reduce or eliminate proliferation of the tumor or other transformed cell in the subject.

6. The method according to claim 5, wherein the subject is infected by a cancer causing virus.

7. A method of treating a subject at least infected with a microbe or bearing a tumor or other transformed cell, said method comprising administration of a pharmaceutical composition comprised of poly(I:C₁₁₋₁₄U) in an amount sufficient to induce dendritic cell maturation.

8. A method of vaccinating a subject against a microbe or tumor, said method comprising administration of (i) a vaccine or dendritic cell preparation which induces an immune response against the microbe or tumor and (ii) a pharmaceutical composition comprised an amount of poly(I:C₁₁₋₁₄U) sufficient to bind to Toll-Like Receptor 3 (TLR3) and to stimulate the immune response against a microbial or tumor antigen of the vaccine or dendritic cell preparation in the subject.

9. The method according to claim 3, wherein the microbe or the cancer or other transformed cell is susceptible to the sole action of poly(I:C₁₁₋₁₄U) acting exclusively as a TLR3 agonist.

10. The method according to claim 3, wherein the microbe or the cancer or other transformed cell is susceptible to the specific cytokine response pattern activated by poly(I:C₁₁₋₁₄U) acting exclusively as a TLR3 agonist.

11. The method according to claim 3, wherein the microbe or the cancer or other transformed cell expresses an antigen that is spontaneously selected by poly(I:C₁₁₋₁₄U) as an in situ target to initiate an immune response against the antigen.

12. The method according to claim 3, wherein at least cytokine production or co-stimulatory molecule signaling which had been initiated by the microbe or the cancer or other transformed cell in the subject is remodulated by poly(I:C₁₁₋₁₄U).

13. The method according to claim 1, wherein the subject is human.

14. The method according to claim 1, wherein poly(I:C₁₂₋₁₄U) is infused intravenously.

15. The method according to claim 1, wherein poly(I:C₁₁₋₁₄U) is injected intradermally, subcutaneously, or intramuscularly; inhaled intranasally or intratracheally; or applied oropharyngeally or sublingually.

16. Use of a mismatched double-stranded ribonucleic acid (dsRNA) to manufacture a medicament for binding to Toll-Like Receptor 3 (TLR3) on immune cells of a subject infected by a microbe, bearing a cancer or other transformed cell, or vaccinated against a microbe, cancer cell, or other transformed cell.
